(19) United States
(12) Patent Application Publication
(10) Pub. No.: US 2024/0115488 A1
(43) Pub. Date: Apr. 11, 2024

(54) COMPOSITION FOR ORAL CAVITY CARE

(71) Applicant: ICHIMARU PHARCOS CO., LTD., Gifu (JP)

(72) Inventor: Ryota TAKEUCHI, Gifu (JP)

(21) Appl. No.: 18/264,930

(22) PCT Filed: Feb. 1, 2022
(86) PCT No.: PCT/JP2022/003836

§ 371 Date: Aug. 10, 2023

(30) Foreign Application Priority Data
Feb. 12, 2021 (JP) 2021-020876
Aug. 31, 2021 (JP) 2021-140997

(51) Int. Cl. A61K 8/9722; A61K 8/34; A61K 8/41; A61K 8/43; A61K 8/49; A61Q 11/00

(52) U.S. Cl. CPC A61K 8/9722; A61K 8/347; A61K 8/416; A61K 8/43; A61K 8/4926; A61Q 11/00; A61K 2800/92

(57) ABSTRACT

An oral care product that may suppress adhesion of bacteria floating in the oral cavity to the teeth by not using an antibacterial ingredient or by reducing the concentration of the antibacterial ingredient and a material contained in the product are produced. A composition for oral cavity care contains a Codium algae extract, and contains, as an active ingredient, a protein fraction contained in the Codium algae extract and having a molecular weight of 5000 or more.

### CROSS-REFERENCE TO RELATED APPLICATIONS

This application is a National Phase of International Application No. PCT/JP2022/003836 filed Feb. 1, 2022, which claims priority to Japanese Patent Application No. 2021-20876 filed on Feb. 12, 2021, and Japanese Patent Application No. 2021-140997 filed on Aug. 31, 2021, in Japan, the entire contents of which are incorporated herein by reference. In addition, all contents described in all patents, patent applications, and documents cited in the present application are incorporated herein by reference in their entirety.

### TECHNICAL FIELD

Embodiments relate to a composition for oral cavity care containing, as an active ingredient, a protein fraction obtained from a Codium algae extract and having a specific molecular weight.

### BACKGROUND ART

A periodontal disease is an inflammatory disease that is developed due to colonization of causative bacteria for the periodontal disease, which are one of oral pathogenic bacteria, in the oral cavity. When the oral cavity is not sufficiently cleaned, dental plaque (oral bacteria and metabolites thereof) adheres to the boundary between the gingiva and the teeth, fixed, and grows. In response to this foreign substance, neutrophils and macrophages are infiltrated, resulting in inflammation. Oral cleaning, such as brushing, at the early stage relieves this inflammation. However, when accumulated dental plaque is left, the inflammation spreads. If a periodontal pocket is formed, dental plaque accumulated in the periodontal pocket is difficult to be removed by brushing or the like. Therefore, suppression of dental plaque, that is, sterilization or bacteriostasis of oral pathogenic bacteria may be useful as a means effective for prevention or amelioration of periodontal disease.

In recent years, dental plaque is considered as a biofilm, bacteria in an oral biofilm (dental plaque) are largely different from floating bacteria in protein expression pattern and drug resistance of bacteria, and it is clear that a drug effective against the floating bacteria is not effective against biofilm-forming bacteria.

It has been known that many antibacterial agents, for example, cationic antibacterial agents such as cetylpyridinium chloride, benzethonium chloride, or chlorhexidine, and nonionic antibacterial agents such as triclosan, are blended in a composition for the oral cavity and are effective as a sterilization means. However, the antibacterial agents do not singly exhibits a sufficient biofilm reduction effect due to the drug resistant mechanism of biofilm bacteria. In order to enhance the sterilization capacity of the antibacterial agents, a improvement technique including combined use of another component has been proposed. However, a significant effect is not obtained due to their low drug permeability into the biofilm or other reasons.

Patent Document 1 discloses that a mushroom-derived lectin ABA that recognizes a sugar chain having terminal Galβ1-3GalNAc and GlcNAc structures has an effect of suppressing attachment and proliferation of oral bacteria onto the plaque or the biofilm in the oral cavity. It has been reported that lectin that is contained in a phycobiont extract of Codium fragile and is bound to a sugar chain that competes with GalNAc may suppress adhesion and proliferation of oral bacteria, especially Streptococcus mutans, which may prevent dental caries (for example, see Patent Document 2).

### CITATION LIST

### Patent Documents

Patent Document 1: Japanese Patent No. 5558362
Patent Document 2: Japanese Patent No. 5925431

### Non-Patent Documents

Non-Patent Document 1: Dental Medicine Research 30 (1): 9-14, 2010
Non-Patent Document 2: Journal of Showa University Dental Society 22: 214-219, 2002

### SUMMARY OF THE INVENTION

### Technical Problem

Use of an oral care product containing an antibacterial ingredient, such as a mouthwash may cause, for example, a side effect, such as diarrhea. Such a side effect usually disappears when taking of an antibacterial agent is stopped. Furthermore, the antibacterial ingredient may reduce nonpathogenic bacteria inherently colonized in the body, thereby giving opportunity for infection with another pathogen. In addition, there is concern about generation of bacteria resistant to the antibacterial ingredient. Therefore, it is strongly desired to provide an oral care product that is used instead of the antibacterial ingredient (including a reduction in the content of the antibacterial ingredient) and a material which can be contained in such an oral care product.

It is an object of embodiments to produce an oral care product that may suppress adhesion of bacteria floating in the oral cavity on the teeth without using an antibacterial ingredient or by reducing the concentration of the antibacterial ingredient, and a material that is contained in the oral care product.

### Solution to the Problem

The embodiments have been made to solve the problems. The embodiments are directed to an oral care product containing a protein fraction contained in a Codium algae extract and having a specific molecular weight, a material contained in the product (oral care material), and the like.

Specifically, aspects of the present invention include the following embodiments.
[1] A composition for oral cavity care, containing, as an active ingredient, a Codium algae extract. The composition contains, as an active ingredient, a protein fraction having a molecular weight of 5000 or more, the protein fraction being contained in the Codium algae extract. The molecular weight of the protein fraction is preferably 5000 or more and less than 50000, further preferably 5000 or more and less than 30000. Alternately, the protein fraction may be a protein in a polymer fraction having a molecular weight of 50000 or more.
[2] The composition of [1], further containing an antibacterial ingredient.
[3] The composition of [1] or [2], wherein an antibacterial ingredient is one or more selected from the group consisting of cetylpyridinium chloride (CPC), chlorhexidine, benzalkonium chloride, benzethonium chloride, dequalinium chloride, chlorhexidine gluconate, protamine, dodecyl di(aminoethyl) glycine, triclosan, 3-methyl-4-isopropyl methylphenol (IPMP), thymol, carvacrol, farnesol, bisabolol, cineole, hinokitiol, sodium lauroyl sarcosinate, and 1-menthol.
[4] The composition of any one of [1] to [3], wherein the active ingredient is contained in an amount of from 0.001 mass % to 5 mass % inclusive relative to a whole amount of the composition in terms of the Codium algae extract.
[5] The composition of any one of [2] to [4], wherein the antibacterial ingredient is contained in an amount of from 0.0001 mass % to 1 mass % relative to a whole amount of the composition.

### Advantages of the Invention

A composition for oral cavity care of the embodiments can suppress adhesion of bacteria flowing in the oral cavity to the teeth without using an antibacterial ingredient or by reducing the concentration of the antibacterial ingredient.

### BRIEF DESCRIPTION OF THE DRAWINGS

FIG. 1 shows results of a bacteria adsorption test for a Codium fragile extract liquid (ML), cetylpyridinium chloride (CPC), and a mixture thereof (CPC+ML).

FIG. 2 shows results of a bacteria adsorption test for a Codium fragile extract liquid (ML), benzalkonium chloride, and a mixture thereof (benzalkonium chloride+ML).

FIG. 3 shows results of a bacteria adsorption test for a Codium fragile extract liquid (ML), chlorhexidine, and a mixture thereof (chlorhexidine+ML).

FIG. 4 shows results of a bacteria adsorption test for a Codium fragile extract liquid (ML), IPMP, and a mixture thereof (IPMP+ML).

FIG. 5 shows results of a bacteria adsorption test for four fractions obtained by molecular weight fractionation in Example 2 (the molecular weights of 50000 or more, 30000 or more and less than 50000, 5000 or more and less than 30000, and less than 5000).

FIG. 6 shows results of a bacteria adsorption test for three fractions obtained by molecular weight fractionation in Example 2 (the molecular weights of less than 5000, 5000 or more and less than 30000, and 30000 or more and less than 50000).

FIG. 7 shows results of a bacteria adsorption test for four fractions obtained by molecular weight fractionation in Example 2 (the molecular weights of 50000 or more, 30000 or more and less than 50000, 5000 or more and less than 30000, and less than 5000) when CPC is added as an antibacterial agent.

FIG. 8 shows results of examinations of a combined effect of a Codium fragile extract liquid and a commercially available mouthwash in a bacteria adsorption test using the Codium fragile extract liquid and the mouthwash.

### DESCRIPTION OF EMBODIMENT

Hereinafter, embodiments of the present invention will be described with reference to the drawings. The embodiments described below are each not limited to the invention according to claims. All components described in the embodiments and combinations thereof are not necessarily essential to solution according to the present invention.

(Composition for Oral Cavity Care)

Herein, a “composition for oral cavity care” is, for example, as follows.
A product (oral care product) that is, in common use, not intentionally swallowed for the purposes of systemic administration of a particular therapeutic agent, but rather retained in the oral cavity for a time sufficient to contact substantially all of the dental surfaces and/or oral tissues for the purposes of oral activity.
A material contained in the product. The material contains a Codium algae extract.

The oral care product may be in a form of a mouthwash, a mouth rinse, a dentifrice, a toothpaste, a gel, a solution, or another form. The oral care product may be incorporated into floss, strips, or films for direct application or adhesion to the oral surface or incorporated into a device or applicator, such as a toothbrush or a roll-on applicator. Such an applicator may be for single or multiple use.

A composition for oral cavity care according to one of embodiments of the present invention contains, as an active ingredient, a Codium algae extract or a protein fraction contained in the Codium algae extract and having a molecular weight of 5000 or more. The details will be described below.

(Codium Algae Extract)

The Codium algae extract used in embodiments of the present invention (hereinafter referred to as the “extract of the embodiment”) may be an extract from alga belonging to the family Codiaceae, the order Codiales (in the recent classification, may be classified into Bryopsidales), the class Chlorophyceae. Examples of alga include Codium fragile, Codium tomentosum, Codium minus, Codium spongiosum, Codium subtubulosum, Codium intricatum, Codium adhaerens, Codium arabicum, Codium coactum, Codium barbatum, Codium contractum, Codium latum, and Codium cylindricum, but is not limited thereto, and alga may belong to biological species of the genus Codium.

A method for collecting the extract of the embodiments may be any method as long as activity for suppressing or inhibiting binding of oral bacteria to a biopolymer in saliva is not substantially impaired.

The extract of the embodiment may be collected from any tissues of phycobiont. The phycobiont may be cleaned, then crushed with a homogenizer or the like, followed by freeze-drying into a powder form or pulverized into a powder form with a pulverizer, and subjected to extraction. An aqueous solution used in the extraction includes: an aqueous solution containing NaCl or another salt, such as saline; a mixture of a water-soluble organic solvent such as acetone and/or an alcohol, and water; and purified water or deionized water, but not limited thereto. The aqueous solution used can be any aqueous solvent well known to those skilled in the art. Herein, the alcohol includes ethanol, ethylene glycol, butylene glycol, and glycerol, but not limited thereto. The aqueous solvent may have an acidic or alkaline pH. In order to adjust the pH of the aqueous solvent, the aqueous solvent contains adipic acid, citric acid, gluconic acid, succinic acid, acetic acid, tartaric acid, lactic acid, fumaric acid, malic acid, phosphoric acid, potassium carbonate, sodium carbonate, sodium bicarbonate, or a potassium or sodium salt of phosphate, but not limited thereto. In order to keep the pH constant, the aqueous solvent contains a tris-hydrochloride buffer, a Hepes buffer, a phosphate buffer, an acetate buffer, a citrate buffer, or a glycine-hydrochloride buffer, but not limited thereto. An extraction method can be appropriately selected from an immersion extraction method, a pressurization extraction method, a supercritical or subcritical extraction method, and the like, or a combination thereof can be used. Extraction conditions may be any conditions as long as activity for suppressing or inhibiting binding of oral bacteria to a biopolymer in saliva is not substantially impaired, but an extraction time is preferably from 10 minutes to 24 hours, and an extraction temperature may be 4° C. or higher, preferably equal to or higher than room temperature, more preferably 15° C. or higher.

(Active Ingredient)

Next, an active ingredient for the composition for oral cavity care can be obtained from the extract of the embodiment as it is or by purification or concentration of the extract. The active ingredient is in a protein fraction having a molecular weight of 5000 or more, more preferably 5000 or more and less than 50000, yet more preferably 5000 or more and less than 30000.

The purification and concentration of the Codium algae extract can be carried out by one of or a combination of any two or more of techniques including, but not limited to, centrifugal separation, salt precipitation, dialysis, vacuum concentration, ultrafiltration, gel filtration, ion exchange chromatography, affinity chromatography, and the like. After the purification or concentration, the Codium algae extract may be vacuum-dried or diluted with a solvent, and then used, if necessary.

As a candidate for the active ingredient of the embodiments, for example, lectin to be bound to a sugar chain that competes with GalNAc is considered (see Patent Document 2). However, the active ingredient is not necessarily bound to all sugar chains having a terminal GalNAc. A second or third sugar residue from the terminal is bound only to a sugar chain having a particular structure, but the binding may be inhibited in the presence of GalNAc.

Herein, lectin refers to a protein having an ability of specifically binding to a sugar chain, other than an antibody involved in the immune system of animals, a T-cell receptor, a Toll-like receptor, and other immune proteins. Lectin can generally aggregate erythrocytes or other animal cells. According to Kanji Hori (KAGAKU TO SEIBUTSU, 32: 586-594 (1994)), lectins from Codium fragile are bound to a sugar chain that competes with GalNAc in common. Although not wishing to be bound by any theory, it is believed that the active ingredient of the embodiments is bound to GalNAc present on the surface of a film formed from polysaccharides contained in a biopolymer in saliva, thereby inhibiting adhesion oral bacteria to the surface.

The concentration of the active ingredient of the embodiments may be any concentration as long as activity for suppressing or inhibiting binding of oral bacteria to a biopolymer in saliva is not substantially impaired, but is preferably 0.001 mass % or more, more preferably 0.01 mass % or more, and yet more preferably 0.1 mass % or more in terms of the Codium fragile extract, relative to the whole amount of the composition for oral cavity care. The upper limit of the concentration is 5 mass % or less, preferably 4 mass % or less, more preferably 3 mass % or less, and yet more preferably 2 mass % or less.

The concentration of a protein contained in the active ingredient of the embodiments may be any concentration as long as activity for suppressing or inhibiting binding of oral bacteria to a biopolymer in saliva is not substantially impaired, and is preferably 0.5 μg/mL or more, more preferably 5 μg/mL or more.

(Antibacterial Ingredient)

The composition for oral cavity care of this embodiment contains a predetermined amount of antibacterial ingredient. The antibacterial ingredient is an component demonstrating antibacterial activity. For example, the antibacterial activity is sterilizing activity, bactericidal activity, disinfecting activity, bacteriostasis, bacteriostatic activity, decolonization activity, aseptic activity, antimicrobial activity, or antifungal activity. An antibacterial agent is an agent containing the antibacterial ingredient. Examples of the antibacterial agent include a bactericide, an antibacterial agent, a fungicide, an antifungal agent, an antiseptic, a deodorant, and a pesticide. Among them, a microorganism controlling agent such as a bactericide, an antibacterial agent, a fungicide, or an antifungal agent, or a deodorant can be suitably used. As the antibacterial ingredient contained in the composition for oral cavity care of the embodiment, a publicly known compound demonstrating the above-described activity may be appropriately selected and used.

Examples of a publicly known antibacterial agent include an organic synthetic antibacterial agent, a natural product-derived antibacterial agent, and an inorganic substance-derived antibacterial agent. For example, the antibacterial ingredient contained in the antibacterial agent is cetylpyridinium chloride (CPC), chlorhexidine, benzalkonium chloride, benzethonium chloride, dequalinium chloride, chlorhexidine gluconate, protamine, dodecyl di(aminoethyl) glycine, triclosan, 3-methyl-4-isopropyl methylphenol (IPMP), thymol, carvacrol, farnesol, bisabolol, cineole, hinokitiol, sodium lauroyl sarcosinate, 1-menthol, or the like. They may be used alone or in combination of two or more of them. In consideration of intended use and antibacterial activity, the concentration of the antibacterial ingredient used is appropriately determined depending on the type of the antibacterial ingredient. In the case of using a cationic antibacterial agent, for example, the antibacterial ingredient can be used within the range of from 0.00001 mass % to 0.5 mass %, preferably from 0.0001 mass % to 0.01 mass %. In the case of using an acidic antibacterial agent, the antibacterial ingredient is used within the range of from 0.01 mass % to 0.5 mass %. In the case of using a polyol having 5 to 6 carbon atoms, the concentration of the antibacterial ingredient is preferably within the range of from 0.1 mass % to 5.0 mass %, more preferably from 1.0 mass % to 4.0 mass %.

The standards for manufacturing marketing approval of external preparations for the oral cavity that affirm indications on medicinal toothbrushing products (Notification No. 0325 of the Pharmaceutical and Food Safety Bureau, No. 37, Mar. 25, 2015, the Director of the Pharmaceutical and Food Safety Bureau, Ministry of Health, Labour and Welfare) specify the type, specifications, and amount of usable active ingredient depending on the indications. For example, it is described that the concentration of chlorhexidine hydrochloride blended as an antibacterial ingredient usable for prevention of periodontitis (alveolar pyorrhea) is from 0.001 mass % to 0.05 mass %. It is described that, as an antibacterial ingredient usable for prevention of gingivitis, the concentration of cetylpyridinium chloride blended is from 0.01 mass % to 0.05 mass %, the concentration of benzalkonium chloride blended is 0.01 mass %, and the concentration of isopropyl methylphenol blended is from 0.02 mass % to 0.1 mass % (see Table 1 attached to the standards for manufacturing marketing approval (Notification No. 0325 of the Pharmaceutical and Food Safety Bureau, No. 37, Mar. 25, 2015, the Director of the Pharmaceutical and Food Safety Bureau, Ministry of Health, Labour and Welfare)). The composition for oral cavity care of the embodiment contains the antibacterial agent having the predetermined specifications in the predetermined amount. Therefore, the composition for oral cavity care can enhance an effect of suppressing or inhibiting binding of oral bacteria to a biopolymer in saliva and simultaneously demonstrate bactericidal activity or bacteriostasis against the oral bacteria. Alternatively, the antibacterial agent may be contained in a decreased amount that is equal to or less than the amount (concentration) described herein. In this case, while a side effect of the antibacterial agent is reduced, the effect of suppressing or inhibiting binding of oral bacteria to a biopolymer in saliva can be enhanced.

Although not wishing to be bound by any theory of a synergistic effect between the Codium algae extract and the antibacterial agent, a synergistic effect between the Codium algae extract and an ionic antibacterial agent is higher than a synergistic effect between the Codium algae extract and a nonionic antibacterial agent. Therefore, it is believed that the active ingredient of the embodiments and the antibacterial agent form a composite in some way to further enhance lectin activity.

Alternatively, it is believed that the active ingredient of the embodiments inhibits an interaction between a biopolymer from saliva, particularly a sugar chain having terminal galactose and the oral bacteria. There is also a possibility that the presence of the antibacterial ingredient together changes the cell membrane structure of the oral bacteria to reduce the binding force of the lectin-like substance derived from the oral bacteria present on the membrane surface.

In any case, the activity of suppressing or inhibiting binding of oral bacteria to a biopolymer in saliva in the Codium algae extract is enhanced in the presence of the antibacterial ingredient having an extremely low concentration. Thus, this fact may be used as a means for solving various problems that are caused by a biofilm formed by microorganisms. For example, it is pointed out in the medical field that a biofilm formed on the surface of a catheter causes a severe infection. A medical device treated with a composition containing the Codium algae extract and the antibacterial ingredient may suppress generation of a biofilm on the surface of the medical device.

Hereinafter, the embodiments will be described further in detail by Examples. However, the embodiments are not limited to Examples. In the following Examples, a unit % of a numerical value indicating the amount of each component added means mass % (w/v %).

### EXAMPLES

### [Example 1] Preparation of Codium Algae Extract (Codium Fragile Extract Liquid)

As a raw material, Codium fragile phycobiont was used. As the raw material, undried raw algae was purchased from a commercial grower. The raw algae purchased was dried and pulverized by a predetermined method, and used as the raw material. To 50 g of the raw material, Dulbecco's phosphate buffered saline (pH 7.3, D5652, Sigma-Aldrich Co. LLC., without Ca and Mg, hereinafter referred to as “PBS”) was added in an amount ten times the amount of the raw material, and the mixture was allowed to stand at room temperature for 30 minutes to prepare a Codium fragile extract liquid. The extract liquid was centrifuged at 37000×g for 15 minutes, and a supernatant was collected. The protein concentration of the extract liquid was measured with a BCA protein assay kit (Thermo Fisher Scientific Inc.). A two-fold dilution series of the extract liquid with PBS was prepared, and subjected to an experiment of evaluating an effect on adhesion and proliferation of Streptococcus mutans.

### [Example 2] Fractionation of Codium Fragile Extract Liquid

10 mL of the Codium fragile extract liquid prepared in Example 1 was treated with Vivaspin 20 (manufactured by Sartorius) of fractionated molecular weights of 50 k, 30 k, and 5 k in turn. Four fractions having molecular weights of less than 5000, 5000 or more and less than 30000, 30000 or more and less than 50000, and 50000 or more were obtained. The protein concentration of each of the four fractions was measured with a BCA protein assay kit (Thermo Fisher Scientific Inc.). The fractions were each diluted with PBS to have a predetermined concentration, and then subjected to the following bacteria adsorption test, and the like.

### [Test Example 1] Bacteria Adsorption Test

<Preparation of Bacterial Culture>

Streptococcus mutans (NBRC13955) was purchased from National Institute of Technology and Evaluation (NITE). The Streptococcus mutans was cultured in Todd-Hewitt Broth at 37° C. overnight (approximately 16 hours), and proliferated until the absorbance OD₆₆₀ measured at 660 nm reached to a value in the range from 0.8 to 1.4. The Streptococcus mutans proliferated was used. Based on the absorbance measured, the Streptococcus mutans was diluted with PBS such that OD₆₆₀ was 0.125, to prepare a bacterial culture to be used in the following test.

<Preparation of Evaluation Sample>

The protein concentration of the Codium fragile extract liquid prepared in Example 1 was 765 μg/mL. The Codium fragile extract liquid was diluted with PBS to have a concentration of 1%, and then used in the following test.

Each antibacterial agent used in the test was prepared to have a concentration that was two times a test concentration for mixing of the antibacterial agent with the Codium fragile extract liquid, a fraction having a predetermined molecular weight, or PBS at 1:1.

As a CPC solution, a stock solution (2% CPC) was obtained by dissolving 0.2 g of CPC in 10 mL of ethanol solution. This solution was diluted 20-fold with purified water to prepare a 0.1% CPC (5% ethanol) solution. This solution was further diluted with a 5% ethanol solution to prepare 0.05% CPC (5% ethanol) and 0.02% CPC (5% ethanol).

As a benzalkonium chloride solution, a stock solution (2% benzalkonium chloride) was obtained by dissolving 0.2 g of benzalkonium chloride in 10 mL of ethanol solution. This solution was diluted 5-fold with ethanol to prepare an ethanol solution of 0.4% benzalkonium chloride solution. This solution was diluted 20-fold with purified water to prepare 0.02% benzalkonium chloride (5% ethanol). This was further diluted 2-fold with a 5% ethanol solution to prepare a 0.01% benzalkonium chloride (5% ethanol) solution. This solution was diluted 2 times with a 5% ethanol solution to prepare a 0.005% benzalkonium chloride (5% ethanol) solution.

A 20% aqueous chlorhexidine solution was used as an undiluted solution of chlorhexidine solution. The chlorhexidine solution was diluted 20-fold with ethanol and purified water to prepare a 0.1% chlorhexidine (5% ethanol) solution. This solution was further diluted 5-fold with a 5% ethanol solution to prepare a 0.02% chlorhexidine (5% ethanol) solution. This solution was further diluted 10-fold with 5% ethanol to prepare a 0.002% chlorhexidine (5% ethanol) solution.

As an IPMP solution, a stock solution (4% IPMP) was obtained by dissolving 0.4 g of IPMP in 10 mL of ethanol solution. This solution was diluted 20-fold with ethanol and purified water to prepare a 0.2% IPMP (20% ethanol) solution. This solution was further diluted 2-fold with 20% ethanol to prepare a 0.1% IPMP (20% ethanol) solution. This solution was further diluted with 20% ethanol to prepare a 0.04% IPMP (20% ethanol) solution.

<Bacteria Adsorption Test>

Two or more hours after toothbrushing, saliva was collected. Stimulated saliva secreted by mastication of Parafilm was collected, and then centrifuged at 4° C. and 2,000×g for 30 minutes. A supernatant was filtered with suction through a cellulose mixed ester membrane filter (0.1 μm, 90 mm), and the saliva was diluted with PBS to have an appropriate concentration.

100 μL of the human saliva prepared as describe above was added to a 96-well multiplate (made from polystyrene, MultiSorp, manufactured by Thermo Fisher Scientific Inc.), the multiplate was sealed, and incubation was carried out at 37° C. for 1 hour. Each well was then cleaned twice with 300 μL of PBS, 100 μL of each of the samples described above was added, the multiplate was sealed, and incubation was carried out at 37° C. for 1 hour. Subsequently, the liquid was discarded from each of the wells, the wells were cleaned twice with 300 μL of PBS, and 100 μL of the bacterial culture prepared such that OD₆₆₀ was 0.125 was added to each well. The multiplate was sealed, and incubation was carried out at 37° C. for 16 hours. The bacterial culture was discarded from each well of the multiplate, the wells were cleaned twice with 300 μL of PBS, 100 μL of 0.25% glutaraldehyde solution was added, and fixation was carried out at room temperature for 30 minutes. Subsequently, the liquid was discarded, and 100 μL of 0.1% crystal violet solution was added to each well and allowed to stand at room temperature for 30 minutes. The stain solution was aspirated and discarded with a pipet, and the multiplate was sufficiently cleaned with purified water and then dried in a constant temperature oven set at 37° C. 100 μL of 30% acetic acid solution was added to each well, and the dye was eluted using a plate shaker. In quantitative determination of the number of bacterial cells, the dye concentration of the 30% acetic acid solution in each well was measured at a light absorption wavelength of 570 nm using a plate reader.

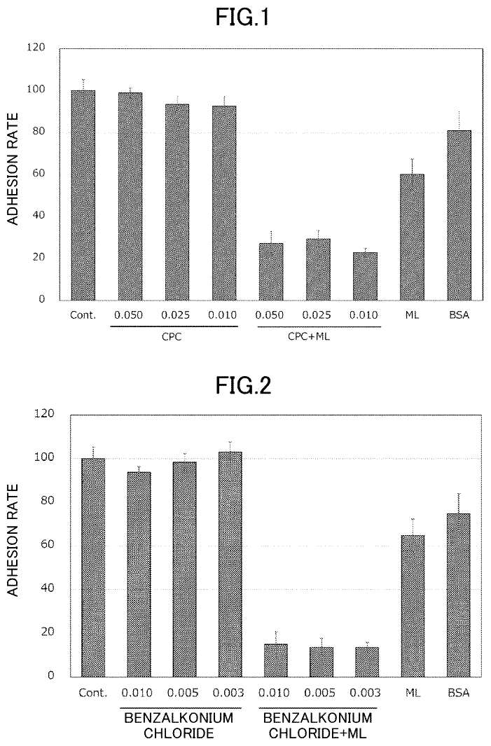

The results are shown in FIGS. 1 to 7. FIG. 1 is a graph showing a suppression effect of the Codium fragile extract liquid (ML), cetylpyridinium chloride (CPC), and a mixture thereof (CPC+ML) on adhesion of Streptococcus mutans to a substrate coated with saliva. An adhesion rate in the graph indicates an average value of values repeatedly measured 5 times under the same conditions. In the graph, the smaller the adhesion rate is, the more the adhesion of Streptococcus mutans is suppressed. A Cont. group shows a measurement result of a sample containing only PBS. A BSA group on a horizontal axis is a group in which 100 μg/mL bovine serum albumin (BSA) was added as a positive control. FIG. 1 shows a relative value of each sample when the value of the Cont. group is set as 100. The numerical value (adhesion rate) of each group shown in FIG. 1 is shown in Table 1.

TABLE 1

| | Adhesion |
|---|---|
| Each group | rate |
| Cont. | 100 |
| Group in which 0.050 (w/v %) CPC was added | 98.9 |
| (indicated as CPC 0.050 in FIG. 1) | |
| Group in which 0.025 (w/v %) CPC was added | 93.4 |
| (indicated as CPC 0.025 in FIG. 1) | |
| Group in which 0.010 (w/v %) CPC was added | 92.6 |
| (indicated as CPC 0.010 in FIG. 1) | |
| Group in which 0.050 (w/v %) CPC and 1.0 w/v % | 27.3 |
| Codium fragile extract liquid were added (indicated | |
| as CPC 0.050 + ML in FIG. 1) | |
| Group in which 0.025 (w/v %) CPC and 1.0 w/v % | 29.4 |
| Codium fragile extract liquid were added (indicated | |
| as CPC 0.025 + ML in FIG. 1) | |
| Group in which 0.010 (w/v %) CPC and 1.0 w/v % | 22.9 |
| Codium fragile extract liquid were added (indicated | |
| as CPC 0.010 + ML in FIG. 1) | |
| Group in which 1.0 w/v % Codium fragile extract | 60 |
| liquid was added (indicated as ML in FIG. 1) | |
| Group in which 100 μg/mL BSA was added (indicated | 80.9 |
| as BSA in FIG. 1) | |

As shown in FIG. 1 and Table 1, the adhesion rate in a group (ML) in which the 1.0 w/v % Codium fragile extract liquid was added was significantly lower than that in the Cont. group (in the Student's t test, p<0.01). As shown in FIG. 1 and Table 1, the adhesion rates in groups (CPC 0.050+ML, CPC 0.025+ML, and CPC 0.010+ML) in which predetermined amounts of CPC and the Codium fragile extract liquid were added were significantly lower than that in the group (ML) in which the 1.0 w/v % Codium fragile extract liquid was added (in the Student's test, p<0.01).

FIG. 2 similarly shows test results when benzalkonium chloride was used as an antibacterial ingredient instead of CPC. FIG. 2 shows a relative value of each sample when the value of the Cont. group is set as 100. The numerical value (adhesion rate) of each group shown in FIG. 2 is shown in Table 2.

TABLE 2

| | Adhesion |
|---|---|
| Each group | rate |
| Cont. | 100 |
| Group in which 0.050 (w/v %) benzalkonium chloride | 93.8 |
| was added (indicated as benzalkonium chloride 0.050 | |
| in FIG. 2) | |
| Group in which 0.025 (w/v %) benzalkonium chloride | 98.4 |
| was added (indicated as benzalkonium chloride 0.025 | |
| in FIG. 2) | |
| A group to which 0.010 (w/v %) of benzalkonium | 103 |
| chloride was added (indicated as benzalkonium | |
| chloride 0.010 in FIG. 2). | |
| Group in which 0.050 (w/v %) benzalkonium chloride | 15 |
| and 1.0 w/v % Codium fragile extract liquid were | |
| added (indicated as benzalkonium chloride 0.050 + | |
| ML in FIG. 2) | |
| Group in which 0.025 (w/v %) benzalkonium chloride | 13.7 |
| and 1.0 w/v % Codium fragile extract liquid were | |
| added (indicated as benzalkonium chloride 0.025 + | |
| ML in FIG. 2) | |
| Group in which 0.010 (w/v %) benzalkonium chloride | 13.6 |
| and 1.0 w/v % Codium fragile extract liquid were | |
| added (indicated as benzalkonium chloride 0.010 + | |
| ML in FIG. 2) | |
| Group in which 1.0 w/v % Codium fragile extract | 64.8 |
| liquid was added (indicated as ML in FIG. 2) | |
| Group in which 100 μg/mL BSA was added (indicated | 74.8 |
| as BSA in FIG. 2) | |

As shown in FIG. 2 and Table 2, the adhesion rate in a group (ML) in which the 1.0 w/v % Codium fragile extract liquid was added was significantly lower than that in the Cont. group (in the Student's t test, p<0.01). As shown in FIG. 2 and Table 2, the adhesion rates in groups (benzalkonium chloride 0.010+ML, benzalkonium chloride 0.005+ML, and benzalkonium chloride 0.003+ML) in which predetermined amounts of benzalkonium chloride and the Codium fragile extract liquid were added were significantly lower than that in the group (ML) in which the 1.0 w/v % Codium fragile extract liquid was added (in the Student's test, p<0.01).

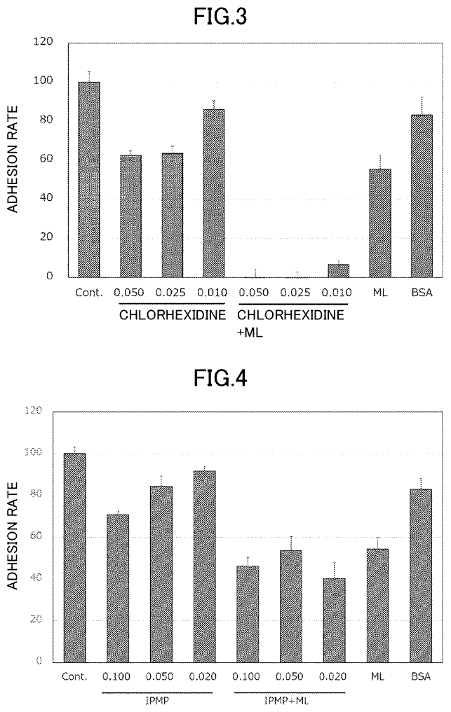

FIG. 3 shows similarly test results when chlorhexidine was used as an antibacterial ingredient instead of CPC or benzalkonium chloride. FIG. 3 shows a relative value of each sample when the value of the Cont. group is set as 100. The numerical value (adhesion rate) of each group shown in FIG. 3 is shown in Table 3.

TABLE 3

| | Adhesion |
|---|---|
| Each group | rate |
| Cont. | 100 |
| Group in which 0.050 (w/v %) chlorhexidine was | 62.4 |
| added (indicated as chlorhexidine 0.050 in FIG. 3) | |
| Group in which 0.025 (w/v %) chlorhexidine was | 63.3 |
| added (indicated as chlorhexidine 0.025 in FIG. 3) | |
| Group in which 0.010 (w/v %) chlorhexidine was | 85.9 |
| added (indicated as chlorhexidine 0.010 in FIG. 3) | |
| Group in which 0.050 (w/v %) chlorhexidine and 1.0 | 0 |
| w/v % Codium fragile extract liquid were added | |
| (indicated as chlorhexidine 0.050 + ML in FIG. 3) | |
| Group in which 0.025 (w/v %) chlorhexidine and 1.0 | 0 |
| w/v % Codium fragile extract liquid were added | |
| (indicated as chlorhexidine 0.025 + ML in FIG. 3) | |
| Group in which 0.010 (w/v %) chlorhexidine and 1.0 | 6.6 |
| w/v % Codium fragile extract liquid were added | |
| (indicated as chlorhexidine 0.010 + ML in FIG. 3) | |
| Group in which 1.0 w/v % Codium fragile extract | 55.3 |
| liquid was added (indicated as ML in FIG. 3) | |
| Group in which 100 μg/mL BSA was added | 83 |
| (indicated as BSA in FIG. 3) | |

As shown in FIG. 3 and Table 3, the adhesion rate in a group (ML) in which the 1.0 w/v % Codium fragile extract liquid was added was significantly lower than that in the Cont. group (in the Student's t test, p<0.01). As shown in FIG. 3 and Table 3, the adhesion rates in groups (chlorhexidine 0.050+ML, chlorhexidine 0.025+ML, and chlorhexidine 0.010+ML) in which predetermined amounts of chlorhexidine and the Codium fragile extract liquid were added were significantly lower than that in the group (ML) in which the 1.0 w/v % Codium fragile extract liquid was added (in the Student's test, p<0.01).

FIG. 4 similarly shows test results when IPMP was used instead of the antibacterial agent. FIG. 4 shows a relative value of each sample when the value of the Cont. group is set as 100. The numerical value (adhesion rate) of each group shown in FIG. 4 is shown in Table 4.

TABLE 4

| | Adhesion |
|---|---|
| Each group | rate |
| Cont. | 100 |
| Group in which 0.100 (w/v %) IPMP was | 70.7 |
| added (indicated as IPMP 0.100 in FIG. 4) | |
| Group in which 0.050 (w/v %) IPMP was | 84.3 |
| added (indicated as IPMP 0.050 in FIG. 4) | |
| Group in which 0.020 (w/v %) IPMP was | 91.5 |
| added (indicated as IPMP 0.020 in FIG. 4) | |
| Group in which 0.100 (w/v %) IPMP and 1.0 | 46.1 |
| w/v % Codium fragile extract liquid were | |
| added (indicated as IPMP 0.100 + ML in FIG. 4) | |
| Group in which 0.050 (w/v %) IPMP and 1.0 | 53.5 |
| w/v % Codium fragile extract liquid were | |
| added (indicated as IPMP 0.050 + ML in FIG. 4) | |
| Group in which 0.020 (w/v %) IPMP and 1.0 | 40.2 |
| w/v % Codium fragile extract liquid were | |
| added (indicated as IPMP 0.020 + ML in FIG. 4) | |
| Group in which 1.0 w/v % Codium fragile extract | 54.3 |
| liquid was added (indicated as ML in FIG. 4) | |
| Group in which 100 μg/mL BSA was added | 82.7 |
| (indicated as BSA in FIG. 4) | |

As shown in FIG. 4 and Table 4, the adhesion rate in a group (ML) in which the 1.0 w/v % Codium fragile extract liquid was added was significantly lower than that in the Cont. group (in the Student's t test, p<0.01). As shown in FIG. 4 and Table 4, the adhesion rates in groups (IPMP 0.100+ML, IPMP 0.050+ML, and IPMP 0.020+ML) in which predetermined amounts of IPMP and the Codium fragile extract liquid were added were significantly lower than that in the group (ML) in which the 1.0 w/v % Codium fragile extract liquid was added (in the Student's test, p<0.01).

FIG. 5 shows results of the bacteria adsorption test of the Codium fragile extract liquid in Example 1 (extract liquid not fractionated) and the four fractions of the Codium fragile extract liquid obtained by molecular weight fractionation in Example 2 (molecular weights of less than 5000, 5000 or more and less than 30000, 30000 or more and less than 50000, and 50000 or more) under the same conditions as described above using a sample having a protein concentration of 10 μg/mL. In FIG. 5, data in which a significant difference (p<0.01) relative to Control is recognized in the Student's t test are represented by **. The numerical value (adhesion rate) of each group shown in FIG. 5 is shown in Table 5.

TABLE 5

| | Adhesion |
|---|---|
| Each group | rate |
| Control (group containing no Codium fragile extract | 100 |
| liquid) | |
| Group in which Codium fragile extract liquid in | 33.6 |
| Example 1 was added at protein concentration of 10 | |
| μg/mL (indicated as no fractionation in FIG. 5) | |
| Group in which fraction having molecular weight of | 133.6 |
| less than 5000 of Codium fragile extract liquid | |
| was added at protein concentration of 10 μg/mL | |
| (indicated as less than 5k in FIG. 5) | |
| Group in which fraction having molecular weight of | 88.2 |
| 5000 or more and less than 30000 of Codium fragile | |
| extract liquid was added at protein concentration of | |
| 10 μg/mL (indicated as 5k or more and less than 30k | |
| in FIG. 5) | |
| Group in which fraction having molecular weight of | 121.6 |
| 30000 or more and less than 50000 of Codium fragile | |
| extract liquid was added at protein concentration | |
| of 10 μg/mL (indicated as 30k or more and less than | |
| 50k in FIG. 5) | |
| Group in which fraction of Codium fragile extract | 11.1 |
| liquid having molecular weight of 50000 or more | |
| was added at protein concentration of 10 μg/mL | |
| (indicated as 50k or more in FIG. 5) | |

As shown in FIG. 5 and Table 5, it was confirmed that the adhesion rates of bacteria in a group of the Codium fragile extract liquid (no fractionation) in Example 1, a group of the fraction having a molecular weight of 50,000 or more of the Codium fragile extract liquid (50 k or more), and a group of the fraction having a molecular weight of 5000 or more and less than 30,000 of the Codium fragile extract liquid were low. Analysis was carried out by increasing the protein concentration in groups of three fractions of the Codium fragile extract liquid (molecular weights of less than 5000, 5000 or more and less than 30000, and 30000 or more and less than 50000) in which a low adhesion rate was not confirmed in the experiment involved in FIG. 5 and Table 5.

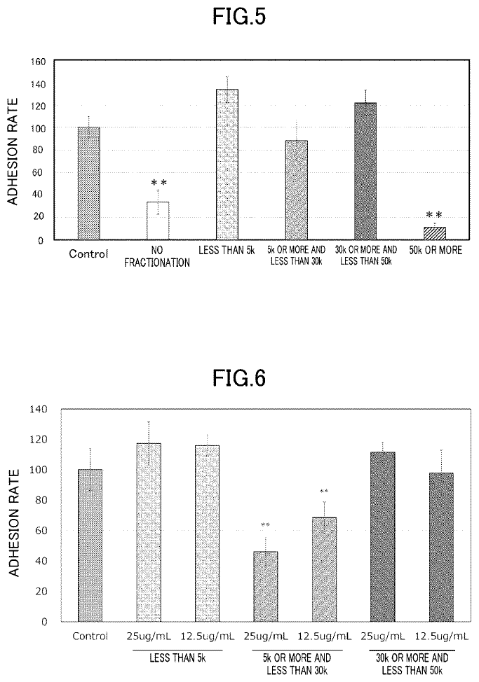

The three fractions of the Codium fragile extract liquid were subjected to a bacteria adsorption test using a high concentration of sample (diluted with a two-fold dilution series from a protein concentration of 50 μg/mL) for confirmation of concentration dependency. The results of the test are shown in FIG. 6 and Table 6. In FIG. 6, data in which a significant difference (p<0.01) relative to Control is recognized in the Student's t test are represented by **. The numerical value (adhesion rate) of each group shown in FIG. 6 is shown in Table 6.

TABLE 6

| | Adhesion |
|---|---|
| Each group | rate |
| Control (group containing no Codium fragile | 100 |
| extract liquid) | |
| Group in which fraction having molecular weight | 117.3 |
| of less than 5000 of Codium fragile extract liquid | |
| was added at protein concentration of 25 μg/mL | |
| (indicated as less than 5k at 25 μg/mL in FIG. 6) | |
| Group in which fraction having molecular weight | 115.9 |
| of less than 5000 of Codium fragile extract liquid | |
| was added at protein concentration of 12.5 μg/mL | |
| (indicated as less than 5k at 12.5 μg/mL in FIG. 6) | |
| Group in which fraction having molecular weight | 46.1 |
| of 5000 or more and less than 30000 of Codium | |
| fragile extract liquid was added at protein | |
| concentration of 25 μg/mL (indicated as 5k or | |
| more and less than 30k at 25 μg/mL in FIG. 6) | |
| Group in which fraction having molecular weight | 68.4 |
| of 5000 or more and less than 30000 of Codium | |
| fragile extract liquid was added at protein | |
| concentration of 12.5 μg/mL (indicated as 5k or | |
| more and less than 30k at 12.5 μg/mL in FIG. 6) | |
| Group in which fraction having molecular weight | 111.6 |
| of 30000 or more and less than 50000 of Codium | |
| fragile extract liquid was added at protein | |
| concentration of 25 μg/mL (indicated as 30k or | |
| more and less than 50k at 25 μg/mL in FIG. 6) | |
| Group in which fraction having molecular weight | 97.9 |
| of 30000 or more and less than 50000 of Codium | |
| fragile extract liquid was added at protein | |
| concentration of 12.5 μg/mL (indicated as 30k or | |
| more and less than 50k at 12.5 μg/mL in FIG. 5) | |

As shown in FIG. 6 and Table 6, a decrease in the adhesion rate in dependence on the protein concentration was confirmed in a group of the fraction having a molecular weight of 5000 or more and less than 30000 of the Codium fragile extract liquid.

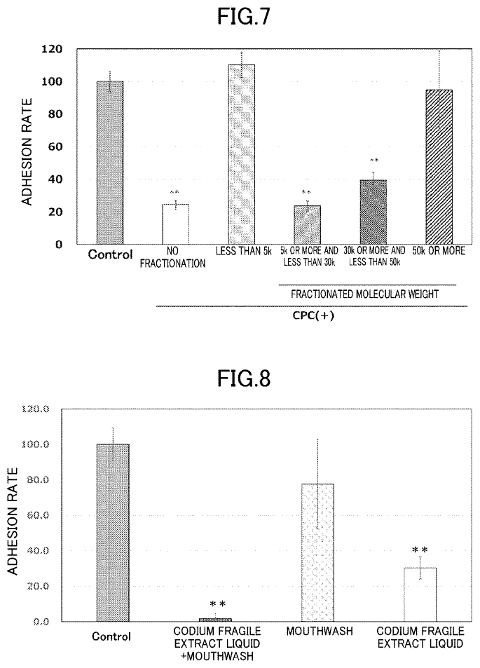

For the Codium fragile extract liquid in Example 1 (extract liquid not fractionated) and the four fractions of the Codium fragile extract liquid obtained by molecular weight fractionation in Example 2 (molecular weights of less than 5000, 5000 or more and less than 30000, 30000 or more and less than 50000, and 50000 or more), CPC (antibacterial ingredient) was added to have a final concentration of 0.05%, and the same bacteria adsorption test was conducted. Each sample of the Codium fragile extract liquid in Example 1 and the fractionated Codium fragile extract liquids was added to have a final concentration of 1.5%. The test results are shown in FIG. 7 and Table 7. In FIG. 7, data in which a significant difference (p<0.01) relative to Control is recognized in the Student's t test are represented by **.

TABLE 7

| | Adhesion |
|---|---|
| Each group | rate |
| Control (group containing no Codium fragile extract | 100 |
| liquid and CPC) | |
| Group in which Codium fragile extract liquid in | 24.4 |
| Example 1 was added (indicated as no fractionation | |
| and CPC(+) in FIG. 7) | |
| Group in which fraction having molecular weight of | 110.2 |
| less than 5000 of Codium fragile extract liquid was | |
| added (indicated as less than 5k CPC(+) in FIG. 7) | |
| Group in which fraction having molecular weight of | 23.5 |
| 5000 or more and less than 30000 of Codium fragile | |
| extract liquid was added (indicated as 5k or more | |
| and less than 30k at 25 μg/mL CPC(+) in FIG. 7) | |
| Group in which fraction having molecular weight of | 39.4 |
| 30000 or more and less than 50000 of Codium fragile | |
| extract liquid was added at protein concentration of | |
| 25 μg/mL (indicated as 30k or more and less than 50k | |
| CPC(+) in FIG. 7) | |
| Group in which fraction having molecular weight of | 94.8 |
| 50000 or more of Codium fragile extract liquid was | |
| added (indicated as 50k or more CPC(+) in FIG. 7) | |

As shown in FIG. 7 and Table 7, the adhesion rates at least in a group of no fractionation, a group of 5 k or more and less than 30 k, and a group of 30 k or more and less than 50 k were confirmed to be lower than that in a Control group.

### [Test Example 2] Bacteria Adsorption Test

An effect and the like in the case where the Codium fragile extract liquid in Example 1 and a commercially available mouthwash were used in combination were checked by a bacteria adsorption test in the same manner as in Test Example 1, using the Codium fragile extract liquid and the mouthwash. The Codium fragile extract liquid in Example 1 was added to have a final concentration of 1.0%. In Test Example 2, GUM Dental Rinse (regular, SUNSTAR) was used as the commercially available mouthwash. Ingredients contained in the mouthwash are as follows.

[Ingredients Contained in the Mouthwash]

Solvent: concentrated glycerin, ethanol/flavouring agent: fragrance (mint type), sodium saccharin/solubilizing agent: POE hydrogenated castor oil/medicinal ingredient: cetylpyridinium chloride (bactericide CPC), dipotassium glycyrrhizinate (anti-inflammatory agent GK2), benzalkonium chloride (bactericide BKC)/pH adjuster: sodium citrate, citric anhydride/cleaning assistant: coconut oil fatty acid acyl arginine ethyl/DL-PCA salt

The confirmed results are shown in FIG. 8 and Table 8. In FIG. 8, data in which a significant difference (p<0.01) relative to Control is recognized in the Student's t test are represented by **.

TABLE 8

| | Adhesion |
|---|---|
| Each group | rate |
| Control (group containing no Codium fragile extract | 100 |
| liquid and CPC) | |
| Group in which Codium fragile extract liquid in | 1.7 |
| Example 1 and mouthwash were added (indicated as | |
| Codium fragile extract liquid + mouthwash in FIG. 8) | |
| Group in which fraction having molecular weight of | 110.2 |
| less than 5000 of Codium fragile extract liquid was | |
| added (indicated as less than 5k CPC(+) in FIG. 7) | |
| Group in which fraction having molecular weight of | 23.5 |
| 5000 or more and less than 30000 of Codium fragile | |
| extract liquid was added (indicated as 5k or more | |
| and less than 30k at 25 μg/mL CPC(+) in FIG. 7) | |

As shown in FIG. 8 and Table 8, the adhesion rates in at least a group in which the Codium fragile extract liquid was added and a group in which the Codium fragile extract liquid and the mouthwash were added were confirmed to be lower than that in Control.

### [Test Example 3] Confirmation of Halitosis Suppression Effect

The presence or absence of a halitosis inhibition effect of a predetermined Codium algae extract liquid and the like was confirmed. Hereinafter, a test method and the like will be described.

<Preparation of Test Sample>

(Preparation of Predetermined Codium Fragile Extract Liquid)

For preparation of a Codium algae extract containing a protein having a molecular weight of 5000 or more (the extract does not contain a protein having a molecular weight of less than 5000, hereinafter referred to as “ML with 5000 or more”), 10 mL of the Codium fragile extract liquid prepared in Example 1 was prepared. Using Vivaspin 20 (manufactured by Sartorius) of a fractionated molecular weight of 5 k, ML with 5000 or more was prepared.

For preparation of three samples described below, 10 mL of the Codium fragile extract liquid prepared in Example 1 was prepared.
A Codium algae extract containing a protein having a molecular weight of 5000 or more and less than 30000 (the extract does not contain a protein other than the protein having a molecular weight of 5000 or more and less than 30000, hereinafter referred to as “ML with 5000 or more and less than 30000”)
A Codium algae extract containing a protein having a molecular weight of 30000 or more and less than 50000 (the extract does not contain a protein other than the protein having a molecular weight of 30000 or more and less than 50000, hereinafter referred to as “ML with 30000 or more and less than 50000”)
A Codium algae extract containing a protein having a molecular weight of 50000 or more (the extract does not contain a protein other than the protein having a molecular weight of 50000 or more, hereinafter referred to as “ML with 50000 or more”)
10 mL of the Codium fragile extract liquid prepared in Example 1 was treated using Vivaspin 20 (manufactured by Sartorius) of fractionated molecular weights of 50 k, 30 k, and 5 k in turn. Three fractions including ML with 5000 or more and less than 30000, ML with 30000 or more and less than 50000, and ML with 50000 or more were obtained.

(Preparation of Predetermined CPC Solution)

A predetermined CPC solution was prepared as follows. 0.2 g of CPC was dissolved in 10 mL of ethanol solution to prepare a stock solution (2% CPC). This stock solution was diluted 20-fold with purified water to prepare a 0.1% CPC (5% ethanol) solution. This solution was further diluted with a 5% ethanol solution to prepare 0.05% CPC (5% ethanol), 0.025% CPC (5% ethanol), and 0.0125% CPC (5% ethanol).

(Preparation of Sample Used in Measurement of VSC Concentration (Preparation of Oral Bacteria Culture Medium))

Saliva (approximately 2 mL) of a healthy human (male) in his 30 s was collected by a predetermined method. The collected saliva was centrifuged (2000×g, 10 minutes, 25° C. (room temperature)). After the centrifugation, the supernatant was collected. The supernatant was added to 30 mL of Todd Hewitt Broth (Becton, Dickinson and Company, 249240), and cultured at 37° C. for 16 hours. The turbidity (OD660 nm) of the culture medium after the culture was measured, and the culture medium was diluted with PBS to have a turbidity of 0.125. The diluted liquid (oral bacteria culture medium) was used in a test.

<Measurement of VSC Concentration>

Halitosis is defined as “malodor that exceeds a socially acceptable limit among gases that come out of the mouth or the nose”. Most (80% or more) of the halitosis is considered to be from gas in the oral cavity, and main causative substances thereof are considered to be hydrogen sulfide (H₂S), methyl mercaptan (CH₃SH), and dimethyl sulfide [(CH₃)₂S], which are volatile sulfur compounds (VSCs) (Cause and fact of halitosis, e-healthnet, Ministry of Health, Labour and Welfare, URL: https://www.e-healthnet.mhlw.go.jp/information/teeth/h-07-001.html). In this Test Example 3, a halitosis suppression effect of a predetermined Codium fragile extract liquid and the like was checked by measuring a VSC concentration.

(Preparation of Plate for VSC Measurement)

100 μL of the human saliva prepared was added to a 96-well multiplate (made from polystyrene, MultiSorp, manufactured by Thermo Fisher Scientific Inc.), the multiplate was sealed, and incubation was carried out at 37° C. for 1 hour, as described in Test Example 1. Subsequently, the wells were cleaned twice with 300 μL of PBS. The following preparations were carried out to use 5 wells for each group listed in Table 9.

After the cleaning, 100 μL of each of the samples listed in Table 9 was added in each of the following Test Examples (Test Examples 3-1, 3-2, and 3-3), the multiplate was sealed, and incubation was carried out at 37° C. for 1 hour. Subsequently, the liquid was discarded from each of the wells, and the wells were cleaned twice with 300 μL of PBS.

After the cleaning, 100 μL of the oral bacteria culture medium was added to each of the wells. The multiplate was sealed, and incubation was carried out at 37° C. for 16 hours. The bacterial culture was discarded from each well of the multiplate, and the wells were cleaned twice with 300 μL of PBS.

After the cleaning, 100 μL of 3.3 mM cysteine solution was added to each of the wells, the multiplate was sealed, and incubation was carried out at 37° C. for 1 hour.

A glass vial (AS ONE Corporation, LABORAN screw tube bottle, No. 3, 9 mL, 9-852-05) in which 3 M phosphoric acid (250 μL, reaction terminator, KISHIDA CHEMICAL Co., Ltd., 260-61925) was placed was prepared. To the glass vial, the liquid obtained by the addition of the cysteine for each group and the incubation (500 μL (=100 μL×5) in total)) was added.

TABLE 9

| Test name | Each group | Explanation of each group |
|---|---|---|
| Test 3-1 | Group 1 | Group in which 1% ML with MW of 5000 |
| | | or more and 0.05% CPC were added |
| | Group 2 | Group in which 0.5% ML with MW of |
| | | 5000 or more and 0.05% CPC were added |
| | Group 3 | Group in which 0.25% ML with MW of |
| | | 5000 or more and 0.05% CPC were added |
| | Group 4 | Group in which 0.05% CPC was added |
| | 3-1 Control | Group containing no Codium fragile |
| | group | extract liquid or CPC solution |
| Test 3-2 | Group 1 | Group in which 0.05% CPC was added |
| | Group 2 | Group in which 0.05% CPC and ML with |
| | | MW of 5000 or more and less than |
| | | 30000 were added |
| | Group 3 | Group in which 0.05% CPC and 1% ML |
| | | with MW of 30000 or more and less |
| | | than 50000 were added |
| | Group 4 | Group in which 0.05% CPC and 1% ML |
| | | with MW of 50000 or more were added |
| | 3-2 Control | Group containing no Codium fragile |
| | group | extract liquid and CPC solution |
| Test 3-3 | Group 1 | Group in which 0.05% CPC and 1% ML |
| | | with MW of 5000 or more were added |
| | Group 2 | Group in which 0.025% CPC and 1% ML |
| | | with MW of 5000 or more were added |
| | Group 3 | Group in which 0.0125% CPC and 1% ML |
| | | with MW of 5000 or more were added |
| | Group 4 | Group in which 0.05% CPC was added |
| | 3-3 Control | Group containing no Codium fragile |
| | group | extract liquid and CPC solution |

A sample for each group was prepared as listed in Table 9, and then allowed to stand at 25° C. (room temperature) for 10 minutes for each group of Test 3-1, each group of Test 3-2, and each group of Test 3-3.

After the standing, the VSC concentration in each group was measured three times with a halimeter (RH17K, TAIYO), and an average value thereof was calculated. The average value of the results (numerical values) obtained was calculated. The calculated results are shown in Table 10.

Table 10 shows relative values (for example, the following values) with respect to an average value in each control group in the groups of Test 3-1, the groups of Test 3-2, and the groups of Test 3-3 that is set as 100.
Groups of Test 3-1: in calculation results, the relative value with respect to the value in the control group of 3-1 is shown, and “*” represents “p<0.05” as compared with the control group of 3-1 in the Student's t test.
Groups of Test 3-2: in calculation results, the relative value with respect to the value in the control group of 3-2 is shown, and “**” represents “p<0.05” as compared with the control group of 3-2 in the Student's t test.
Groups of Test 3-3: in calculation results, the relative value with respect to the value in the control group of 3-3 is shown, and “***” represents “p<0.05” as compared with the control group of 3-3 in the Student's t test.

TABLE 10

| | | | Calculation | |
|---|---|---|---|---|
| Test name | Each group | Explanation of each group | result | |
| Test 3-1 | Group 1 | Group in which 1% ML with MW of 5000 or more | 17.8 | (*) |
| | | and 0.05% CPC were added | | |
| | Group 2 | Group in which 0.5% ML with MW of 5000 or | 28.4 | (*) |
| | | more and 0.05% CPC were added | | |
| | Group 3 | Group in which 0.25% ML with MW of 5000 or | 40.5 | (*) |
| | | more and 0.05% CPC were added | | |
| | Group 4 | Group in which 0.05% CPC was added | 59.1 | (*) |
| | 3-1 Control | Group containing no Codium fragile extract | 100 | |
| | group | liquid and CPC solution | | |
| Test 3-2 | Group 1 | Group in which 0.05% CPC was added | 59.1 | (**) |
| | Group 2 | Group in which 0.05% CPC and ML with MW of | 42.7 | (**) |
| | | 5000 or more and less than 30000 were added | | |
| | Group 3 | Group in which 0.05% CPC and 1% ML with MW of | 25.0 | (**) |
| | | 30000 or more and less than 50000 were added | | |
| | Group 4 | Group in which 0.05% CPC and 1% ML with MW of | 66.1 | (**) |
| | | 50000 or more were added | | |
| | 3-2 Control | Group containing no Codium fragile extract | 100 | |
| | group | liquid and CPC solution | | |
| Test 3-3 | Group 1 | Group in which 0.05% CPC and 1% ML with MW of | 38.8 | (***) |
| | | 5000 or more were added | | |

| | | |
|---|---|---|
| Group 2 | Group in which 0.025% CPC and 1% ML with MW of | 58.4 |
| | 5000 or more were added | |
| Group 3 | Group in which 0.0125% CPC and 1% ML with MW | 65.3 |
| | of 5000 or more were added | |
| Group 4 | Group in which 0.05% CPC was added | 91 |
| 3-3 Control | Group containing no Codium fragile extract | 100 |
| group | liquid and CPC solution | |

As shown in Table 10, the halitosis suppression effect of the predetermined Codium fragile extract liquid was confirmed.

### [Test Example 4] Pigmentation Test Using Apatite Particles

For example, esthetics is required for a dental prosthetic crown used for the anterior teeth (Non-Patent Documents 1 and 2). Thus, suppression of pigmentation using apatite particles was tested using a composition (solution) containing the Codium fragile extract liquid prepared in Example 1 and CPC. This test was carried out as described below based on the descriptions of Non-Patent Documents 1 and 2.

<Test Method>

Reagents and the like are as listed below.
Apatite particles: Bio RAD CHT Ceramic Hydroxyapatite Type I (80 μm), Cat. #158-8000, Lot. M401076
Coffee: commercially available canned coffee, CRAFT BOSS BLACK (Suntory Foods Limited)
Measurement device for color difference measurement: CR-400, KONICA MINOLTA, INC., chroma meter
Saliva to be added in the following test step (saliva solution 1): was prepared as follows.

The saliva of adult healthy human male (in his 30s) was used. Two or more hours after toothbrushing, saliva was collected by stimulation of paraffin gum. The collected saliva was centrifuged (4° C., 2000 g, 30 min), and the supernatant was collected. The supernatant was diluted with PBS to prepare a diluted solution. The diluted solution was filtered through a cellulose mixed ester membrane filter (0.1 μm, 90 mm). The diluted solution after the filtration was used as saliva to be added in the following test step.
Oral bacteria culture medium to be added in the following test step: The saliva (approximately 2 mL) of a healthy human (male) in his 30s was collected by a predetermined method. The collected saliva was centrifuged (2000×g, 10 minutes, 25° C. (room temperature)). After the centrifugation, the supernatant was collected. The supernatant was added to 30 mL of Todd Hewitt Broth (Becton, Dickinson and Company, 249240), and cultured at 37° C. for 16 hours. The turbidity (OD660 nm) of the culture medium after the culture was measured, and the culture medium was diluted with PBS to have a turbidity of 0.125. The diluted culture medium was used as the oral bacteria culture medium to be added in the following test step.
CPC: CPC used in Test Example 1

100 mg of apatite particles was weighed in a 1.5-mL tube. To the tube containing the particles, 1 mL of PBS was added, and the mixture was stirred at 25° C. with a vortex mixer. After the stirring, spinning down was carried out, and the supernatant was discarded from the tube.

After the discarding, 1 mL of the saliva was added to the tube, and the mixture was stirred at 25° C. with a vortex mixer. After the stirring, the mixture was allowed to stand at 37° C. for 1 hour. After the standing, spinning down was carried out, and the supernatant was then discarded from the tube.

The particles in the tube were rinsed twice by adding 1 mL of PBS to the tube (rinsing including vortexing, spinning down, and discarding of the supernatant).

In each of the following groups (Groups 4-1, 4-2, and 4-3), 1 mL of each sample listed in Table 11 was added to the tube, and the mixture was stirred at 25° C. with a vortex mixer. After the stirring, the mixture was allowed to stand at 37° C. for 1 hour. After the standing, spinning down was carried out, and the supernatant was then discarded from the tube.

TABLE 11

| Each group | Added sample |
|---|---|
| Group 4-1 | PBS was added |
| Group 4-2 | 0.01% CPC and 1% Codium fragile extract |
| | liquid prepared in Example 1 were added |
| Group 4-3 | PBS was added |

After the supernatant was discarded, the particles in the tube were rinsed twice by adding 1 mL of PBS to the tube (rinsing including vortexing, spinning down, and discarding of the supernatant).

In Groups 4-2 and 4-3, 1 mL of the “oral bacteria culture medium” was added to the tube, and the mixture was stirred with a vortex mixer. In Group 4-1, the “oral bacteria culture medium” was not added. After the stirring, the mixture was allowed to stand at 37° C. for 1 hour. After the standing, spinning down was carried out, and the supernatant was then discarded from the tube.

The particles in the tube were rinsed twice by adding 1 mL of PBS to the tube (rinsing including vortexing, spinning down, and discarding of the supernatant).

1 mL of the coffee was added to the tube, and the mixture was stirred with a vortex mixer. After the stirring, the mixture was allowed to stand at 37° C. for 1 hour. After the standing, spinning down was carried out, and the supernatant was then discarded from the tube. The particles in the tube were rinsed three times by adding 1 mL of purified water to the tube (rinsing including vortexing, spinning down, and discarding of the supernatant).

After the rinsing three times, the particles in each of the groups were transferred into a petri dish. After the transferring into the petri dish, the particles were dried at 50° C. for 2 hours. After the drying, a color difference in each of the groups was measured with the measurement device. During the measurement, a background color was white. The particles were placed on a standard white board, and the measurement was carried out. The color of the particles was measured before the addition of the coffee, and the value of measured color was a reference value.

For color specification, L*a*b* color system (CIE1976 L*a*b* uniform color space) was used. A L* value represents brightness, and is from 0 to 100. A larger L* value represents brighter color. a*b* represents chromaticness. When a* and b* are zero, the color is achromatic color. As a* is larger in a positive direction, the color is more strongly reddish. As a* is larger in a negative direction, the color is more strongly greenish. As b* is larger in a positive direction, the color is more strongly yellowish. As b* is larger in a negative direction, the color is more strongly bluish. A value of ΔE*, which is used to represent a difference in color, is determined by calculating a direct distance between two colors in this color space.

<Test Results>

The measurement results of color difference are shown in Table 12 below. In Table 12, an average value determined from values measured three times in each group is listed. “*” shown in Table 12 represents “p<0.01” as compared with Group 4-3 in the Student's t test. For the “oral bacteria culture medium”, results (particularly, a result of L* value (brightness)) in Group 4-2 (the group in which the Codium fragile extract liquid and the like were added) are more favorable than those in Group 4-3.

TABLE 12

| | L* value | a* value | b* value |
|---|---|---|---|
| Group 4-1 | 77.87 (*) | 1.74 | 7.51 (*) |
| Group 4-2 | 77.14 (*) | 1.82 | 7.53 (*) |
| Group 4-3 | 76.24 | 1.96 | 8.33 |

### [Test Example 5] Confirmation of Effect of Toothpaste Containing Codium Fragile Extract Liquid

An effect during use of a toothpaste containing the Codium fragile extract liquid prepared in Example 1 in a human oral cavity was confirmed. The effect was confirmed in a test by three adult healthy human males (in their 30s).

The toothpaste containing the Codium fragile extract liquid prepared in Example 1 was prepared according to Formulation Examples 1 to 3 listed in Table 13. As Comparative Example 1, a toothpaste containing no Codium fragile extract liquid listed in Table 13 was prepared. The effect was confirmed by toothbrushing using each of the toothpastes of the Formulation Example 1 to 3 and the Comparative Example 1 listed in Table 13.

TABLE 13

| | | Formulation | Formulation | Formulation | Comparative |
|---|---|---|---|---|---|
| | Contained ingredient | Example 1 | Example 2 | Example 3 | Example 1 |
| Function | Codium fragile extract | 1 | 1 | 1 | 0 |
| Ingredient | CPC | 0.05 | 0.025 | 0.0125 | 0.05 |
| Substrate | Sorbitol liquid (70%) | 35 | 35 | 35 | 35 |
| Ingredient | Propylene glycol | 4 | 4 | 4 | 4 |
| | Polyethylene glycol 400 | 0.1 | 0.1 | 0.1 | 0.1 |
| | Abrasive silica, silicic | 15 | 15 | 15 | 15 |
| | anhydride (abrasive) | | | | |
| | Thickening silica, silicic | 3.5 | 3.5 | 3.5 | 3.5 |
| | anhydride (thickening) | | | | |
| | Xanthan gum | 0.2 | 0.2 | 0.2 | 0.2 |
| | Carrageenan | 0.2 | 0.2 | 0.2 | 0.2 |
| | Polyoxyethylene (20) | 0.5 | 0.5 | 0.5 | 0.5 |
| | hydrogenated castor oil | | | | |
| | Sodium lauryl sulfate | 1 | 1 | 1 | 1 |
| | Sodium polyacrylate | 0.5 | 0.5 | 0.5 | 0.5 |
| | Titanium oxide | 0.4 | 0.4 | 0.4 | 0.4 |
| | Citric acid | Appropriate | Appropriate | Appropriate | Appropriate |
| | | amount | amount | amount | amount |
| | Sodium hydroxide | Appropriate | Appropriate | Appropriate | Appropriate |
| | | amount | amount | amount | amount |
| | Sodium saccharin | 0.15 | 0.15 | 0.15 | 0.15 |
| | Methylparaben | 0.15 | 0.15 | 0.15 | 0.15 |
| | Predetermined perfume | 0.5 | 0.5 | 0.5 | 0.5 |
| | Purified water | Residue | Residue | Residue | Residue |

When the toothpaste of Comparative Example 1 was used, the three males felt refreshed immediately after toothbrushing, but felt sticky in the oral cavity after a certain period of time (after approximately 3 hours). When the toothpaste of the prescription example (Formulation Example 1 to 3) was used, the three males felt refreshed immediately after toothbrushing, and did not feel sticky in the oral cavity after a certain period of time (after approximately 3 hours), unlike the case of using Comparative Example 1.

### [Test Example 6] Confirmation of Effect of Mouthwash Containing Codium Fragile Extract Liquid

An effect during use of a mouthwash containing the Codium fragile extract liquid prepared in Example 1 in a human oral cavity was confirmed. The effect was confirmed in a test by three adult healthy human males (in their 30s).

The mouthwash containing the Codium fragile extract liquid prepared in Example 1 was prepared according to Formulation Example 4 to 6 listed in Table 14. As Comparative Example 2, a mouthwash containing no Codium fragile extract liquid listed in Table 14 was prepared. The effect was confirmed by washing the mouth using each of the mouthwashes of the Formulation Example 4 to 6 and Comparative Example listed in Table 14.

TABLE 14

| | | Formulation | Formulation | Formulation | Comparative |
|---|---|---|---|---|---|
| | | Example 4 | Example 5 | Example 6 | Example 2 |
| Functional | Codium fragile extract | 1 | 1 | 1 | 0 |
| ingredient | CPC | 0.05 | 0.025 | 0.0125 | 0.05 |
| Substrate | Ethanol | 5 | 5 | 5 | 5 |
| ingredient | Polyoxyethylene (60) | 0.5 | 0.5 | 0.5 | 0.5 |
| | hydrogenated castor oil | | | | |
| | Glycerin | 5 | 5 | 5 | 5 |
| | Propylene glycol | 4 | 4 | 4 | 4 |
| | Xylitol | 3 | 3 | 3 | 3 |
| | Citric acid | Appropriate | Appropriate | Appropriate | Appropriate |
| | | amount | amount | amount | amount |
| | Sodium citrate | Appropriate | Appropriate | Appropriate | Appropriate |
| | | amount | amount | amount | amount |
| | Methylparaben | 0.15 | 0.15 | 0.15 | 0.15 |
| | Predetermined perfume | 0.5 | 0.5 | 0.5 | 0.5 |
| | Purified water | Residue | Residue | Residue | Residue |

When the mouthwash of Comparative Example 2 was used, the three males felt refreshed immediately after use of the mouthwash, but felt sticky in the oral cavity after a certain period of time (after approximately 3 hours). When the mouthwash of the formulation examples (Formulation Examples 4 to 6) was used, the three males felt refreshed immediately after use of the mouthwash, and did not feel sticky in the oral cavity after a certain period of time (after approximately 3 hours), unlike the case of using Comparative Example 2.

### INDUSTRIAL APPLICABILITY

By using the composition of the embodiments, it is confirmed that binding of oral bacteria to a biopolymer in saliva can be suppressed or inhibited. Therefore, the composition of the embodiments can be utilized for oral care cosmetics, quasi-drugs, and the like.

1. A composition for oral cavity care, comprising:
a Codium algae extract, wherein
the Codium algae extract contains a protein,
the protein having a molecular weight of 5000 or more, as an active ingredient.

2. The composition of claim 1, further comprising:
an antibacterial ingredient.

3. The composition of claim 2, wherein
the antibacterial ingredient is one or more selected from the group consisting of cetylpyridinium chloride (CPC), chlorhexidine, benzalkonium chloride, benzethonium chloride, dequalinium chloride, chlorhexidine gluconate, protamine, dodecyl di(aminoethyl) glycine, triclosan, 3-methyl-4-isopropyl methylphenol (IPMP), thymol, carvacrol, farnesol, bisabolol, cineole, hinokitiol, sodium lauroyl sarcosinate, and 1-menthol.

4. The composition of claim 1, wherein
the active ingredient is contained in an amount of from 0.001 mass % to 5 mass % relative to a whole amount of the composition in terms of the Codium algae extract.

5. The composition of claim 2, wherein
the antibacterial ingredient is contained in an amount of from 0.0001 mass % to 1 mass % relative to a whole amount of the composition.

6. The composition of claim 2, wherein
the active ingredient is contained in an amount of from 0.001 mass % to 5 mass % relative to a whole amount of the composition in terms of the Codium algae extract.

7. The composition of claim 3, wherein
the active ingredient is contained in an amount of from 0.001 mass % to 5 mass % relative to a whole amount of the composition in terms of the Codium algae extract.

8. The composition of claim 3, wherein
the antibacterial ingredient is contained in an amount of from 0.0001 mass % to 1 mass % relative to a whole amount of the composition.

9. The composition of claim 4, wherein
the antibacterial ingredient is contained in an amount of from 0.0001 mass % to 1 mass % relative to a whole amount of the composition.

10. The composition for oral cavity care, according to claim 1, wherein the Codium algae include Codium fragile, Codium tomentosum, Codium minus, Codium spongiosum, Codium subtubulosum, Codium intricatum, Codium adhaerens, Codium arabicum, Codium coactum, Codium barbatum, Codium contractum, Codium latum, and Codium cylindricum,

11. The composition of claim 1, wherein a concentration of the protein contained in the active ingredient is 0.5 μg/mL or more, more preferably 5 μg/mL or more.

12. The composition of claim 1, wherein a concentration of the protein contained in the active ingredient is 5 μg/mL or more.

13. A method for oral treatment care of suppressing adhesion of bacteria floating in an oral cavity on teeth comprising:
administering to a human a therapeutically effective amount of a composition comprising a Codium algae extract, wherein the Codium algae extract contains a protein,
the protein having a molecular weight of 5000 or more, as an active ingredient.

14. The method of claim 13, further administrating a therapeutically effective amount of an antibacterial ingredient.

15. The method of claim 14, wherein the antibacterial ingredient is one or more selected from the group consisting of cetylpyridinium chloride (CPC), chlorhexidine, benzalkonium chloride, benzethonium chloride, dequalinium chloride, chlorhexidine gluconate, protamine, dodecyl di(aminoethyl) glycine, triclosan, 3-methyl-4-isopropyl methylphenol (IPMP), thymol, carvacrol, farnesol, bisabolol, cineole, hinokitiol, sodium lauroyl sarcosinate, and 1-menthol.

16. The method of claim 13, wherein the active ingredient is contained in an amount of from 0.001 mass % to 5 mass % relative to a whole amount of the composition in terms of the Codium algae extract.

17. The method of claim 14, wherein the antibacterial ingredient is contained in an amount of from 0.0001 mass % to 1 mass % relative to a whole amount of the composition.
